(19) United States
(12) Patent Application Publication
(10) Pub. No.: US 2014/0358141 A1
(43) Pub. Date: Dec. 4, 2014

(54) ELECTROSURGICAL PENCIL WITH A SMOKE EVAPORATION TUBE

(71) Applicants: Jesper Schantz Simonsen, Copenhagen (DK); Niels Kornerup, Rungsted (DK)

(72) Inventors: Jesper Schantz Simonsen, Copenhagen (DK); Niels Kornerup, Rungsted (DK)

(73) Assignee: SAFEAIR AG

(21) Appl. No.: 14/344,291

(22) PCT Filed: Sep. 12, 2012
(86) PCT No.: PCT/EP2012/067849

§ 371 Date: May 16, 2014

(30) Foreign Application Priority Data
Sep. 12, 2011 (DK) PCTDK2011/050339
May 10, 2012 (CN) 201220209711.6
May 10, 2012 (DK) PA 2012 70240

(51) Int. Cl. A61B 18/14; A61B 18/12

(52) U.S. Cl. CPC A61B 18/14; A61B 18/1206; A61B 2018/00601
USPC 606/39

(57) ABSTRACT

An electrosurgical pencil for cutting tissue during surgery by application of electrical energy supplied from a generator. A tube is provided for evacuation of smoke from the site of surgery. To improve flexibility and reduce the stiffness of the tube while maintaining generally the shape and ability of the tube to conduct smoke from the site, the tube comprises a polymer film, herein referred to as sheath stretched by at least one reinforcement element.

### INTRODUCTION

The invention relates to an electrosurgical pencil for electrosurgical procedures such as for cutting tissue during surgery by application of electrical energy supplied from a generator. Particularly, the invention relates to a pencil with an elongated body, an electrode located at a distal end of the body, and a tube extending from an opposite proximal end of the body and facilitating smoke evacuation during surgery.

### BACKGROUND

Electrosurgical instruments have become widely used by surgeons, and a need has developed for equipment and instruments which are safe in use and easy to handle.

By electrosurgical procedure is herein meant a procedure where an electrosurgical instrument transfers radio-frequency (RF) electrical or electrosurgical energy to a tissue site for cutting and/or coagulation of the tissue. In monopolar systems, the electrosurgical energy is returned to the electrosurgical source via a return electrode pad positioned under the patient.

In particular if the pencil is combined with a tube for evacuation of smoke, the surgeon must deal with the presence of not only electrical cables for the RF electrosurgical signal but also with relatively thick tube for the suction. In particular it may be difficult to perform angular adjustment of the surgical electrode inside the body because of the lack of ability of the tube to rotate and to be moved together with the instrument.

### DESCRIPTION OF THE INVENTION

It is an object of the invention to improve the maneuverability of electrosurgical pencils provided with a tube for smoke evacuation.

According to a first aspect, the invention provides a pencil where the tube comprises a polymer sheath stretched by at least one reinforcement element.

Particularly, the reinforcement element may, in a cross-section perpendicular to the direction of conduction by the tube, herein referred to as the axial direction, have a largest dimension being at least 20 times the thickness of the sheath. By this combination between a very thin sheath relative to the reinforcement element, the tube may become highly flexible via a relatively thin sheath, yet highly rigid against collapsing due to the relatively large cross sectional dimension.

In electrosurgical procedures, the pencil is typically handled very carefully. Typically, no abrupt movements are made and the environment is typically not very rough—i.e. there are typically no objects which can be harmful for the tube. Furthermore, the tube is connected to a source of suction, and the consequence of rupturing the tube is not severe—in worst case the smoke evacuation is reduced or stopped. The invention and use of a thin and potentially fragile sheath which is stretched by a reinforcement element or a plurality of elements contradicts a deeply held prejudice governing that the suction tube must withstand tough handling and environment.

Particularly, the invention may provide a pencil where the reinforcement element(s) is at least 10 times the stiffness of the sheath such as at least 100, 500 or 1000 times the stiffness of the sheath.

Due to the large difference in stiffness, the tube may have a very large flexibility—i.e. it may be very easy to bend and form the tube into a desired shape. This flexibility is obtained by elastic deformation of the relatively elastic and/or thin sheath. At the same time, the tube offers a large resistance against collapsing. This resistance is provided by the reinforcement elements which stretch the sheath.

By “stiffness” is herein meant the rigidity of the sheath or reinforcement element, i.e. the resistance of the reinforcement element and sheath against elastic deformation thereof, e.g. as measured by a force being applied and a resulting displacement of the reinforcement element and/or sheath.

The term “electrosurgical pencil” is intended to include an instrument which has a body shaped to fit into the hand of a surgeon and which operates an attached active electrode. The electrode is for an electrosurgical procedure such as for cauterizing, coagulating and/or cutting tissue. Typically, the electrosurgical pencil may be operated by a hand-switch or a foot-switch. The electrode is an electrically conducting element which is usually elongated and may be in the form of a thin flat blade with a pointed or rounded distal end. Alternatively, the electrode may be an elastically deformable element, e.g. a wire or string shaped element, e.g. forming a wire loop by which an electrosurgical procedure may be carried out.

By “tube” is herein meant an oblong element with an inner lumen capable of conducting a fluid flow, the shape and size depending entirely on the need for suction. Herein, the axial direction of the tube is the direction in which the tube conducts a fluid flow, i.e. the lengthwise direction from the body of the pencil to the source providing the suction. In a cross section perpendicular to the axial direction, the tube may e.g. be circular or oval.

By “sheath” is herein meant a thin sheet of a polymer material, e.g. a foil or skin like sheet, e.g. an elastically deformable thin sheet of a polymer material.

The specification of the sheath being stretched by a reinforcement element means that the sheath possesses close to no structural rigidity and is therefore not capable of preserving a specific shape unless being held by the reinforcement element. The word “stretched” herein does not imply any elastic or plastic deformation of the sheath but merely that the sheath is held in a certain shape by the reinforcement element. However, in one embodiment, the sheath may be elastically deformed by the stretching provided by the reinforcement element.

The reinforcement element may form a spiral of convolutions with a certain pitch e.g. in the range of 0.5-2 times a cross sectional dimension of the tube, e.g. 2 times the diameter in case the tube is circular in a cross section perpendicular to the axial direction. The reinforcement element may thereby constitute one single element or a few elements each extending over a larger portion of the tube such that the entire tube comprises a plurality of elements, e.g. 1, 2, 3, 4 or 5 elements for each 5 centimeter.

Within the context of the present invention the term “pitch” means the axial distance between two corresponding points on two adjacent convolutions of a spiral. The convolutions make the medical spiral tube look as if it has an exterior thread or is bellow-like, in relation to which thread or bellow-like shape the term “pitch” is used in the present application. The convolutions coil as a helix along the respective part of the length of the medical spiral tube.

Alternatively, the tube comprises a plurality of reinforcement elements arranged one after the other in axial direction of the tube. In this embodiment, the reinforcement elements may e.g. be ring-shaped, e.g. with a circular shape.

Generally, the tube may be made from a uniform hose formed by the sheath and with the reinforcement element(s) attached circumferentially about the hose or inside the hose. Alternatively, the sheath and reinforcement elements may be manufactured in one and the same integrated manufacturing process, e.g. an extrusion process where the reinforcement elements are inserted into, arranged about, or formed completely integral with the reinforcement elements as the hose is extruded.

Irrespective of the type and number of reinforcement elements, these could be made from a material identical to that of the sheath E.g. from a material comprising PE or from an EVA resin like ELVAX from DuPont or from a similar resin with low vinyl acetate contents or from combinations of such materials.

In an alternative embodiment, the sheath and reinforcement element are made from two different materials, e.g. one from an EVA resin and the other from PE or similar polymer material.

Particularly, the tube may have a cross sectional areal in the range of at least 50 percent of the area of the body of the pencil when seen in a cross section perpendicular to the axial direction of the pencil, e.g. between 60 and 100 percent of the cross sectional area of body of the pencil. Herein, the axial direction of the body is the direction from the distal end of the body to the proximal end of the body.

Additionally, the stiffening element or elements may have a cross sectional area in the range of at least 5-10 percent of the cross sectional area of the body.

Particularly, the reinforcement element(s) may be ring shaped, e.g. circularly ring shaped. In one particular embodiment, the reinforcement element is circular with a diameter being in the range of 50-150 percent of a largest measurable dimension in a cross section perpendicular to the oblong direction of the body, e.g. 75-125 percent of the largest dimension.

It may be an advantage if both the sheath and the reinforcement elements are made from polymer materials and if they have different prevailing direction of the polymer chains. By prevailing direction is herein meant that at least 30 percent of the polymer chains are in essentially the same direction. By essentially the same direction is herein meant that the polymer chains are within plus minus 20 degrees relative to each other such as within plus or minus 10 degrees relative to each other.

The sheath may e.g. have a prevailing direction of the polymer chains in the axial direction, and the reinforcement elements may have a prevailing direction of the polymer chains perpendicular to the axial direction, e.g. a prevailing direction corresponding to the peripheral direction of the reinforcement elements. If the reinforcement elements are ring shaped, e.g. circular, the prevailing direction may be the circular shape of the ring, i.e. at least 30 percent of the polymer chains may extend circularly or along the ring shape.

The sheath may have one or more prevailing directions, e.g. two prevailing directions, e.g. being perpendicular to each other. Particularly, one of the prevailing directions may be in the axial direction of the tube, i.e. in the direction in which the inner lumen of the tube conducts a fluid flow.

The tube may encapsulate conduction cables for supplying the electrode with energy from the generator. This will prevent the conduction cables from getting entangled. In one embodiment, such conduction cables form part of the reinforcement element(s) for the tube. Particularly, if the reinforcement element is spiral shaped, such a spiral shaped element may form a conductor for energy to the electrode. In another embodiment, the reinforcement element(s) may at least be joined to the conduction cable(s) such that the position of the cable(s) inside or outside the tube is fixed by the reinforcement element(s).

The tube could be made by a process comprising the step of providing a tube with a sheath and reinforcement element and subsequently stretching the tube in axial direction to thereby provide an increased distanced between adjacent reinforcement elements or to provide an increased pitch between corresponding points on adjacent convolutions of a spiral shaped reinforcement element. This process may also effectively provide a uniform direction of the polymer chains.

The tube may comprise a proximal section and a distal section where the distance between adjacent reinforcement elements or convolutions of a spiral shaped reinforcement element is different in the proximal and distal sections. Herein, the distal section is directly connected to the body of the pencil and the proximal section is connectable to a source of suction.

The proximal section may have a shorter distance between the adjacent reinforcement elements or a smaller pitch of a spiral convoluted reinforcement element when compared with the distal section.

In one example, a tube with an outer diameter of 13.5 millimeters has a proximal section with a pitch of 6 millimeters and a distal section with a pitch of 4 millimeters. The proximal section thereby becomes more rigid and less fragile. Particularly, the proximal section may better withstand a radial pressure on the tube without collapsing and thereby stopping the fluid flow in the tube. The distal section may, on the other hand, be more flexible and have an improved ability to allow a more free movement of the pencil, including rotation of the pencil.

Since the flexibility to move the pencil is primarily required close to the pencil where the movement and rotation occurs, the distal section may be relatively short compared with the proximal section. The distal section may e.g. have a length being less than 75 percent of the length of the proximal section, e.g. less than 50 percent of the length of the proximal section, such as less than 25 percent of the length of the proximal section.

The tube may have an internal diameter in the range of 5-20 mm, such as 8-15 mm, such as 10-14 mm, and an external diameter in the range of 10-15 mm, such as 12-14 mm. Further, the tube may have a proximal section with a larger diameter than the distal section.

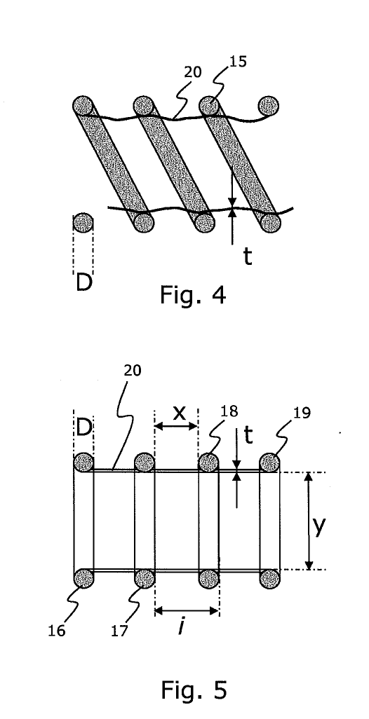

In such a tube, the thickness of the reinforcement element, c.f. the dimension D in FIGS. 4 and 5 later herein could be in the range of 2.5-4 millimeters, such as 2.6-2.8 millimeters.

The thickness of the sheath, c.f. dimension t in FIGS. 4 and 5, may e.g. be in the range of 0.04-0.2 mm, such as in the range of 0.06-1.5 mm, such as in the range of 0.08-1.2 mm.

Particularly, the tube may extend in one piece from the body of the pencil to the source of suction.

The source of suction could be a pump which is connected to the tube by a coupling allowing detachable fixing of the tube to the pump. The coupling may be formed integral with the tube, e.g. by having the tube and coupling being molded in one piece or by bonding the tube and coupling adhesively. Particularly, the coupling may be bonded, or formed in one part with the reinforcement element.

In a second aspect, the invention provides a method of providing smoke evacuation during an electrosurgical procedure, the method comprising using a pencil according to any of claims 1-24. The procedure may particularly imply the use of one single tube connected between the body of the pencil and a source of suction, and particularly to use a tube with a proximal section having a larger distance between adjacent reinforcement elements or a larger pitch.

### DETAILED DESCRIPTION OF THE INVENTION

In the following, embodiments of the invention will be described in further details with reference to the drawing in which:

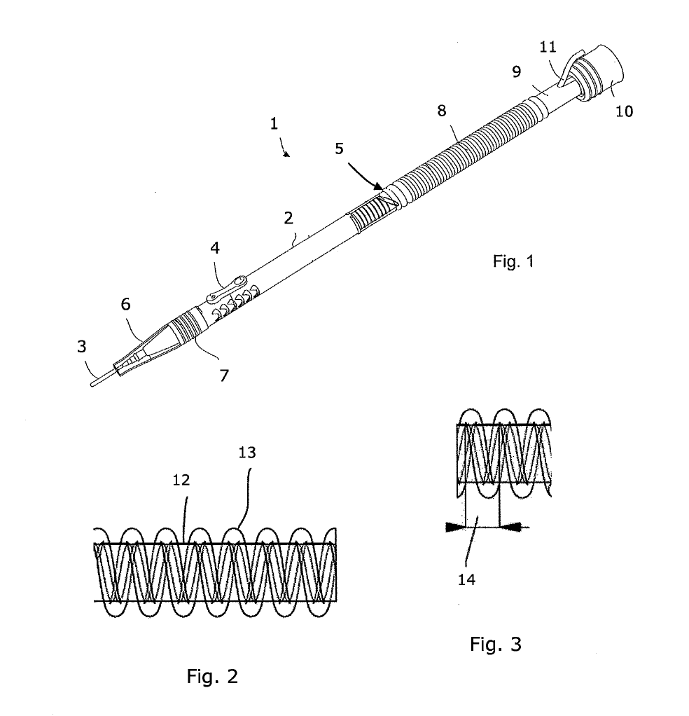

FIG. 1 illustrates a pencil according to the invention;

FIGS. 2-3 illustrate details of the tube for a pencil according to the invention;

FIGS. 4-5 illustrate details of an alternative embodiment of the tube:

FIG. 1 illustrates an electrosurgical pencil for cutting tissue during surgery. In use, electrical energy is supplied from a generator to the electrode. The electrode is attached to a distal end of the elongated body. At an axially opposite, proximal, end of the body, the pencil comprises a fixed or detachably attached tube which facilitates suction and thus removal of smoke from a site around the electrode. The body comprises an internal duct enabling suction through the body when the tube is attached to the proximal end thereof. The duct terminates at the suction tip. The tube is detachable from the body at the location marked by the circle.

FIG. 1 shows a perspective view of the pencil for cutting and/or coagulating tissue of a patient during surgery by application of electrical energy supplied from an electrosurgical generator (not shown).

The pencil 1 comprises a hollow elongated main body 2, a blade electrode 3 which can be used for cutting and/or coagulation. A switch 4 is provided for switching between different settings of the generator.

In the disclosed embodiment, the switch is a rocker switch by which a surgeon can switch between a power mode for cutting and a power mode for coagulation by pressing one of the two ends of the switch 4.

At a proximal end 5, of the body 2, a tube 8 is attached either rigidly or in a removable manner such that the pencil 1 can be used with or without the tube 8. At its opposite end, the pencil comprises a suction tip 6 mounted at a first end 7 of the elongated main body 2 to surround at least a portion of the blade electrode 3.

The suction tube 8 is highly flexible and made of a disposable transparent plastic material. At the distal end of the tube where it is connected to the proximal end of the pencil, the tube comprises a coupling (not shown) fitting into the body 2 of the pencil. In the opposite, proximal, end 9 of the tube, the tube comprises a coupling 10 for attaching the tube to a suction source, i.e. typically a pump which provides a controllable flow of air. The tube is made in one piece between the couplings, and the couplings are joined rigidly to the tube. The tube may alternatively be joined rigidly to the body 2 thereby alleviating the need for the coupling against the body.

A cable 11 is connected to the switch 4 to deliver current from the generator (not shown) to the blade electrode 3 in response to actuation of the switch 4.

In the present embodiment of the electrosurgical instrument 1, the cable 11 extends inside the suction tube 8 towards a proximal end 9 of the suction tube 8, at which proximal end 9 the cable 11 exits the tube 8 just before the coupling 10.

In other embodiments the cable 11 can extend outside the suction tube 8, and in yet other embodiments, the cable 11 may form part of the tube, e.g. form part of a spiral shaped reinforcement element for the tube. In this embodiment, the coupling 10 may comprise socket means mating with socket means in the pump to provide the electrosurgical HF power to the electrode via the pump.

The structure and design of the elongated main body 2 may deviate from the illustrated pen-like shape. Generally, however, it is preferred to provide communication from the tube 11 to the suction tip 6 inside the body.

The body 2 has a substantially semicircular or circular cross-section to make handling easier, not least with respect to rotation of the pencil about an axis defining the longest extend of the pencil.

FIG. 2 illustrates details of the tube 8. The tube comprises a polymer sheath 12 which is held in a correct tubular shape (herein referred to as “stretched”) by at least one reinforcement element 13.

Both the sheath and the reinforcement element are made from a polymer material, e.g. from an EVA resin such as Elvax™ from DuPont, and they are joined during a mutual manufacturing process where the sheath and reinforcement element are made simultaneously. In FIG. 2, the reinforcement element is a spiral shaped element forming convolutions about the tubular sheath.

FIG. 3 illustrates the definition used herein of the term pitch 14, namely the distance between one point on one convolution and the corresponding point on an adjacent convolution.

In a cross section, the reinforcement element may have any shape. However, a circular shape as illustrated in FIGS. 4 and 5 may typically be suitable since it provides a uniform resistance against bending in different directions.

FIGS. 4 and 5 illustrate two different embodiments of the reinforcement element(s) seen in cross sectional views.

In FIG. 4, the tube comprises a single reinforcement element 15 which is spiral shaped, and in FIG. 5, the tube comprises a plurality of individual reinforcement elements 16-19 arranged at a distance in the order of 20-50 percent of the internal diameter, y, of the tube. In the drawing, this is illustrated by the distance x being in the order of 20-50 percent of the distance Y.

The largest dimension of the reinforcement element measured in cross section, (in FIGS. 4 and 5 this corresponds to the diameter D of the reinforcement element) may be in the range of 5-50 times the thickness, t, of the sheath 20, e.g. in the range of 8-12 times the thickness, t, of the sheath.

Please note that the dimensions x and y are illustrated only on FIG. 5. They may, however, indicate the dimension of both of the embodiments of FIGS. 4 and 5.

In the illustrated embodiment in FIG. 5, the repeated period length i has a size in the order of 35-70 percent of the internal diameter y.

The invention relates to the following numbered embodiments:
1. An electrosurgical pencil for cutting tissue during surgery by application of electrical energy supplied from a generator, the pencil comprising an elongated body, an exposed electrode located at a distal end of the body, and a tube extending from an opposite proximal end of the body and facilitating smoke evacuation during surgery, the tube comprising a polymer sheath stretched by at least one reinforcement element.
2. A pencil according to embodiment 1, where the reinforcement element, in a cross-section, has a largest dimension being at least 20 times the thickness of the sheath.
3. A pencil according to embodiment 1 or 2, where each reinforcement element has a stiffness in the range of at least 10 times the stiffness of the sheath.
4. A pencil according to any of the preceding embodiments, where the reinforcement element forms spiral convolutions.
5. A pencil according to embodiment 4, where the spiral convolutions form a pitch in the range of 0.2-2 times a cross sectional dimension of the tube.
6. A pencil according to any of the preceding embodiments, comprising a plurality of reinforcement elements arranged adjacently at a distance of at least 20 percent of a cross sectional dimension of the tube.
7. A pencil according to any of the preceding embodiments, where the stiffening element(s) is made from the same material as the sheath
8. A pencil according to any of the preceding embodiments, where at least one of the stiffening element and the sheath is made from a material selected from the group consisting of PE, EVA, ELVAX™ and combinations thereof.
9. A pencil according to any of the preceding embodiments, where the stiffening element(s) has a cross sectional area of at least 0.15 times the cross sectional area of the body.
10. A pencil according to any of the preceding embodiments, where the sheath comprises a polymeric material with polymer chains having a first prevailing direction.
11. A pencil according to embodiment 10, where the reinforcement element(s) comprises a polymeric material with polymer chains having a second prevailing direction being different from the first prevailing direction.
12. A pencil according to embodiments 10 and 11, where first prevailing direction is essentially perpendicular to the second prevailing direction.
13. A pencil according to any of the preceding embodiments, where the tube encapsulates conduction means for supplying the electrode with energy from a generator.
14. A pencil according to any of the preceding embodiments, where the tube is made by a process comprising the step of providing a tube with a sheath and at least one reinforcement element, and subsequently stretching the tube in axial direction by plastic deformation of the sheath to thereby provide an increased distanced between adjacent reinforcement elements or to provide an increased pitch between corresponding points on adjacent convolutions of a spiral shaped reinforcement element.
15. A pencil according to any of the preceding embodiments, where the tube comprises a proximal section and a distal section and where the distance between adjacent reinforcement elements or convolutions of a spiral shaped reinforcement element is different in the proximal and distal sections.
16. A pencil according to any of the preceding embodiments, where the tube has an internal diameter in the range of 8-12 mm.
17. A pencil according to any of the preceding embodiments, where the tube has an external diameter in the range of 10-14 mm.
18. A pencil according to any of the preceding embodiments, comprising a proximal section and a distal section and where the proximal section has a larger diameter than the distal section.
19. A pencil according to any of the preceding embodiments where the sheath has a thickness in the range of 0.04-0.06 mm.
20. A pencil according to any of the preceding embodiments, where the tube is detachable from the body.
21. A pencil according to any of the preceding embodiments, where the tube has shape memory properties with a shape in a relaxed state by which the axial distance between two corresponding points on two adjacent convolutions of a reinforcement element or on two adjacent reinforcement elements is in the range of 3-9 mm.
22. A pencil according to embodiment 21 where the tube is stretchable by elastic deformation such that a maximum length is in the range of 130-200 percent of the length in the relaxed state.
23. A pencil according to any of the preceding embodiments, where the tube extends in one piece from the body of the pencil to a source of suction.
24. A pencil according to any of the preceding embodiments, comprising a coupling allowing detachable fixing of the tube to a source of suction, the coupling being formed integrally with at least the reinforcement element of the tube.
25. A method of providing smoke evacuation during an electrosurgical procedure, the method comprising using a pencil according to any of embodiments 1-24.

1. An electrosurgical pencil for cutting tissue during surgery by application of electrical energy supplied from a generator, the pencil comprising an elongated body, an exposed electrode located at a distal end of the body, and a tube extending from an opposite proximal end of the body and facilitating smoke evacuation during surgery, the tube comprising a polymer sheath stretched by at least one reinforcement element.

2. A pencil according to claim 1, where the reinforcement element, in a cross-section (D), has a largest dimension being at least 20 times the thickness (t) of the sheath.

3. The pencil according to claim 1, where each reinforcement element has a stiffness in the range of at least 10 times the stiffness of the sheath.

4. The pencil according to claim 1, wherein the reinforcement element forms spiral convolutions.

5. The pencil according to claim 4, wherein the spiral convolutions form a pitch in the range of 0.2-2 times a cross sectional dimension of the tube.

6. The pencil according to claim 1, comprising a plurality of reinforcement elements arranged adjacently at a distance of at least 20 percent of a cross sectional dimension (y) of the tube.

7. The pencil according to claim 1, wherein the stiffening element(s) is made from the same material as the sheath

8. The pencil according to claim 1, where at least one of the stiffening element and the sheath is made from a material selected from the group consisting of PE, EVA, ELVAX™ and combinations thereof.

9. The pencil according to claim 1, wherein the stiffening element(s) has a cross sectional area of at least 0.15 times the cross sectional area of the body.

10. The pencil according to claim 1, wherein the sheath comprises a polymeric material with polymer chains having a first prevailing direction.

11. The pencil according to claim 10, wherein the reinforcement element(s) comprises a polymeric material with polymer chains having a second prevailing direction being different from the first prevailing direction.

12. The pencil according to claim 10, wherein first prevailing direction is essentially perpendicular to the second prevailing direction.

13. The pencil according to claim 1, wherein the tube encapsulates conduction means for supplying the electrode with energy from a generator.

14. The pencil according to claim 1, wherein the tube is made by a process comprising the step of providing a tube with a sheath and at least one reinforcement element, and subsequently stretching the tube in axial direction by plastic deformation of the sheath to thereby provide an increased distanced between adjacent reinforcement elements or to provide an increased pitch between corresponding points on adjacent convolutions of a spiral shaped reinforcement element.

15. The pencil according to claim 1, wherein the tube comprises a proximal section and a distal section and where the distance between adjacent reinforcement elements or convolutions of a spiral shaped reinforcement element is different in the proximal and distal sections.

16. The pencil according to claim 1, wherein the tube has an internal diameter in the range of 8-12 mm.

17. The pencil according to claim 1, wherein the tube has an external diameter in the range of 10-14 mm.

18. The pencil according to claim 1, comprising a proximal section and a distal section and wherein the proximal section has a larger diameter than the distal section.

19. The pencil according to claim 1, wherein the sheath has a thickness in the range of 0.04-0.06 mm.

20. The pencil according to claim 1, wherein the tube is detachable from the body.

21. The pencil according to claim 1, wherein the tube has shape memory properties with a shape in a relaxed state by which the axial distance between two corresponding points on two adjacent convolutions of a reinforcement element or on two adjacent reinforcement elements is in the range of 3-9 mm.

22. The pencil according to claim 21 wherein the tube is stretchable by elastic deformation such that a maximum length is in the range of 130-200 percent of the length in the relaxed state.

23. The pencil according to claim 1, wherein the tube extends in one piece from the body of the pencil to a source of suction.

24. The pencil according to claim 1, comprising a coupling allowing detachable fixing of the tube to a source of suction, the coupling being formed integrally with at least the reinforcement element of the tube.

25. A method of providing smoke evacuation during an electrosurgical procedure wherein the method comprises using a pencil according to claim 1.
